(19) United States
(12) Patent Application Publication
(10) Pub. No.: US 2026/0192101 A1
(43) Pub. Date: Jul. 9, 2026

(54) Urinary Catheter With Bag

(71) Applicant: C. R. Bard, Inc., Franklin Lakes, NJ (US)

(72) Inventors: Ronald N. Legaspi, Alpharetta, GA (US); Pooja Bhuvanesh Kulkarni, Pune (IN); Sudev Geetha Surendran, Kozhikode District (IN); James David Hughett, SR., Monroe, GA (US)

(21) Appl. No.: 19/131,907

(22) PCT Filed: Nov. 21, 2022
(86) PCT No.: PCT/US2022/050648

§ 371 Date: May 21, 2025

(51) Int. Cl. A61M 39/12; A61M 25/00; A61M 39/00; A61M 39/10

(52) U.S. Cl. CPC A61M 39/12; A61M 25/002; A61M 25/0097; A61M 2039/0009; A61M 2039/1027; A61M 2202/0014; A61M 2202/0496; A61M 2205/586; A61M 2209/06; A61M 2210/1085; A61M 2210/1089

(57) ABSTRACT

A urinary catheter system generally configured for self-deployment. The system includes a catheter coupled with a bag. The bag includes a bag cap for sealing the bag when disconnected from the catheter. The bag cap is tethered to the bag and attached to the catheter to secure the bag to catheter. The catheter further includes a sheath covering the catheter tube, where a lumen of the sheath also contains gripping members that grip the catheter tube when a squeezing force is applied to the gripping member. A distal end of the catheter is sealably coupleable to the proximal end after use. A distal end of the catheter is attachable to the proximal end to define a pre-use packaging state. The distal end is also sealably coupleable to the proximal end after use.

### BACKGROUND

Urinary incontinence causes a person to lose control over their bladder resulting in loss of large or small quantities of urine. This can be caused due to multiple reasons. A paraplegic or tetraplegic user loses the control of their bladder and they need to empty their bladder using urinary catheters. These can be indwelling (the catheter is placed for a longer duration inside the bladder and attached to a urine bag to collect the urine) or they can be intermittent (the catheter is periodically inserted into the bladder and the urine is drained from it either directly into appropriate drainage receptacle or into a collection bag). Intermittent catheters are preferred in the cases where the user can self-catheterize or wishes to not be seen with a urine bag. The intermittent catheters allow the user a freedom of mobility.

In some instances, the user may drain his bladder directly into a drainage receptacle, such as a toilet or urinal. In other instances, a drainage receptacle may not be readily accessible. In such instances, the user may connect a bag to the catheter to drain the urine into the bag. In some instances, connecting the bag to the catheter may be inconvenient and may introduce an infection risk to the user. In some instances, a bag disconnected from the catheter may not be resealable, and as such, urine may spill from the bag.

Disclosed herein are urinary catheters and methods of catheterization that address the items described above.

### SUMMARY

Briefly summarized, disclosed herein is a urinary catheter system that, according to some embodiments, includes a catheter coupled with a collection bag. The catheter includes (i) a catheter tube defining a catheter lumen extending between a distal end and a proximal end of the catheter, where the catheter tube is configured for insertion into a bladder of a user and (ii) an outlet hub coupled with the catheter tube at the proximal end, where the outlet hub defines a handle configured for manipulation of the catheter during use. The collection bag includes an inlet hub that defines an opening of the collection bag and an engagement member tethered to the inlet hub. The engagement member is configured to selectively (i) couple with the inlet hub to sealably close off the opening, thereby sealing the collection bag, and (ii) decouple from the inlet hub to provide for access to contents of the collection bag. The inlet hub is coupled with the outlet hub at a proximal opening of the outlet hub to define a fluid flow path between the catheter lumen and an interior of the collection bag. The engagement member is further configured to selectively (i) attach to the outlet hub to prevent separation of the inlet hub from the outlet hub and (ii) detach from the outlet hub to allow separation of the collection bag from the catheter.

In some embodiments, the engagement member is configured to attach to the outlet hub along a side wall of the outlet hub. In further embodiments, the engagement member includes a protrusion, and the outlet hub includes a corresponding depression, where the corresponding depression is configured to receive the protrusion to define the attachment of the engagement member to the outlet hub.

In some embodiments, the catheter further comprises an inlet collar disposed at the distal end of the catheter, and the inlet collar is attached to the outlet hub to define a pre-use packaging state of the catheter.

In some embodiments, the inlet collar includes a clip configured to attach the inlet collar to the outlet hub in the pre-use packaging state.

In some embodiments, the inlet collar is configured to couple with the outlet hub at the proximal opening to define a post-use packaging state of the catheter.

In some embodiments, the catheter further comprises a sheath extending between the outlet hub and the inlet collar, where the sheath defines a sheath lumen, and where the catheter tube is disposed within the sheath lumen.

In some embodiments, the catheter further comprises a number of gripping members disposed within the sheath lumen, where each gripping member is configured to (i) grip the catheter tube in response to a squeezing force applied to the gripping member by a user and (ii) slide along the catheter tube in the absence of the gripping force.

In some embodiments, the catheter further includes a cap configured to couple with the inlet collar, the cap configured to close off a distal end of the inlet collar in the pre-use packaging state.

Also disclosed herein is another embodiment of a urinary catheter system that includes a catheter and a collection bag coupled with the catheter. The catheter includes a catheter tube defining a catheter lumen extending between distal end and a proximal end of the catheter, where the catheter tube is configured for insertion into a bladder of a user. The catheter further includes an outlet hub coupled with the catheter tube at the proximal end, where the outlet hub defines a handle configured for manipulation of the catheter during use. The catheter further includes an inlet collar disposed at the distal end. The collection bag includes an inlet hub defining an opening of the collection bag. The inlet hub is coupled with the outlet hub at a side port of the outlet hub to define a fluid flow path between the catheter lumen and an interior of the collection bag, and the inlet collar is coupled with the outlet hub to define a pre-use packaging state.

In some embodiments, the inlet collar and the outlet hub define a common longitudinal axis in the pre-use packaging state

In some embodiments, the catheter further comprises an engagement member tethered to the inlet hub, and the engagement member configured to selectively (i) couple with the inlet hub to sealably close off the opening, thereby sealing the collection bag, and (ii) attach to the outlet hub to prevent separation of the inlet hub from the outlet hub.

In some embodiments, the catheter further includes a sheath extending between the outlet hub and the inlet collar, where the sheath defines a sheath lumen, and where the catheter tube is disposed within the sheath lumen.

In some embodiments, the catheter further includes a number of gripping members disposed within the sheath lumen, where each gripping member is configured to (i) grip the catheter tube in response to a squeezing force applied to the gripping member by a user and (ii) slide along the catheter tube in the absence of the gripping force.

Also disclosed herein is method for draining urine from a bladder of a user according to some embodiments. The method includes providing a catheter system that includes a catheter coupled with a collection bag, where the catheter includes a catheter tube defining a catheter lumen. The catheter further includes an outlet hub at a proximal end of the catheter, and the collection bag includes an inlet hub, where the outlet hub is coupled with the inlet hub such that the catheter lumen is in fluid communication with an interior of the collection bag. The catheter is attached to the collection bag via a tether, and the outlet hub is attached to an inlet collar of the catheter, where the inlet collar is disposed at distal end of the catheter. The method further includes: (i) detaching the outlet hub from the inlet collar; (ii) advancing the catheter tube of the catheter along a urethra of the user to the bladder of the user to access urine within the bladder; (iii) draining the urine from the bladder, where the urine flows along the lumen of the catheter and into the interior of the bag; (iv) detaching the tether from the catheter; (v) separating the inlet hub from the outlet hub; and (vi) draining the urine from the interior of the bag.

In some embodiments of the method, the tether includes bag cap attached thereto, where the catheter is attached to the bag via the tether includes the bag cap attached to the outlet hub. In such embodiments, detaching the tether from the catheter includes detaching the bag cap from the outlet hub.

In some embodiments, the method further includes attaching the bag cap to the inlet hub to seal the urine within the collection bag.

In some embodiments of the method, advancing the catheter tube includes proximally displacing the inlet collar along the catheter tube.

In some embodiments, the method further includes (i) distally displacing the inlet collar along the catheter tube after draining the urine and (ii) attaching the inlet collar to the outlet hub to define a disposal state of the catheter.

In some embodiments of the method, the catheter further includes: (i) a sheath extending along the catheter tube, where the catheter tube is disposed within a lumen of the sheath; and (ii) a number of gripping members disposed within the lumen of the sheath. Each gripping member is configured to (i) grip the catheter tube in response to a squeezing force applied to the gripping member by a user and (ii) slide along the catheter tube in the absence of the gripping force. In such embodiments, advancing the catheter tube includes applying the squeezing force to at last one gripping member.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustration of a urinary catheter system in a pre-use packaging state, in accordance with some embodiments;

FIG. 1B is an illustration of the urinary catheter system of FIG. 1A in an unpackaged state, in accordance with some embodiments;

FIG. 1C is an illustration of the urinary catheter system of FIG. 1A in ready-to-use state, in accordance with some embodiments;

FIG. 1D is an is an illustration of the urinary catheter system FIG. 1A in use, in accordance with some embodiments;

FIG. 1E is an is an illustration of the urinary catheter system FIG. 1A in a post use state, in accordance with some embodiments;

FIG. 1F is an is an illustration of the urinary catheter system FIG. 1A in a disposal state, in accordance with some embodiments;

FIG. 2A is a detailed illustration of an outlet hub and an inlet hub of the urinary catheter system of FIG. 1A in a coupled state, in accordance with some embodiments;

FIG. 2B is a detailed illustration of an outlet hub and an inlet hub of the urinary catheter system of FIG. 1A in a partially decoupled state, in accordance with some embodiments;

FIG. 2C is a detailed illustration of an outlet hub and an inlet hub of the urinary catheter system of FIG. 1A in a fully decoupled state, in accordance with some embodiments;

FIG. 3A is an illustration of a second embodiment of a urinary catheter system in a pre-use state, in accordance with some embodiments;

FIG. 3B is an illustration of the urinary catheter system of FIG. 3A in a ready-to-use state, in accordance with some embodiments;

FIG. 3C is an illustration of the urinary catheter system of FIG. 3A in a post-use state, in accordance with some embodiments; and

FIG. 3D is an illustration of the urinary catheter system of FIG. 3A in a disposal state, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a user. Likewise, a “proximal length” of, for example, the catheter includes a length of the catheter intended to be near the clinician when the catheter is used on the user. A “proximal end” of, for example, the catheter includes an end of the catheter intended to be near the clinician when the catheter is used on the user. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in user when the catheter is used on the user. Likewise, a “distal length” of, for example, the catheter includes a length of the catheter intended to be near or in the user when the catheter is used on the user. A “distal end” of, for example, the catheter includes an end of the catheter intended to be near or in the user when the catheter is used on the user. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

The phrases “connected to,” “coupled with,” and “in communication with” refer to any form of interaction between two or more entities, including but not limited to mechanical, and fluid interaction. Two components may be coupled with each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

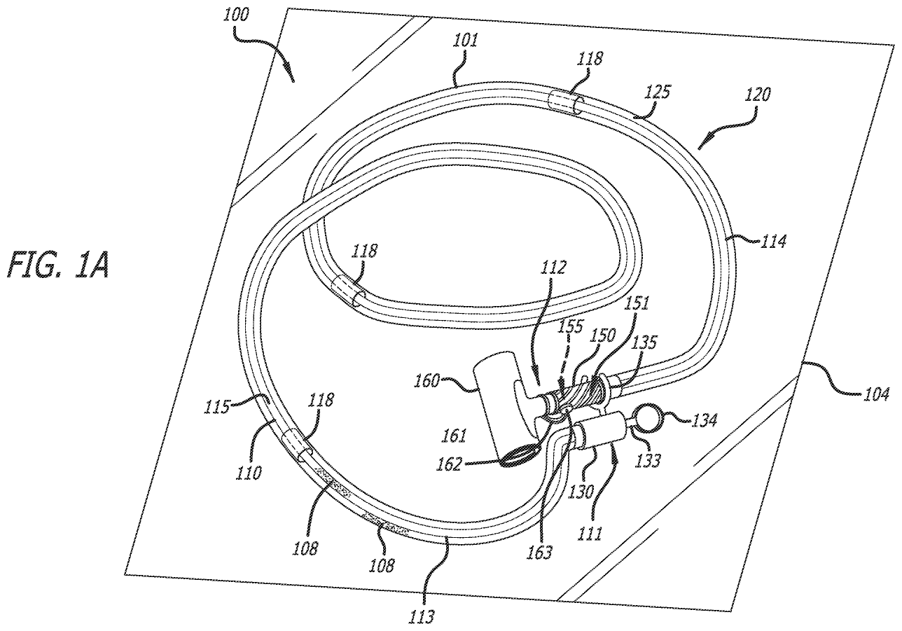

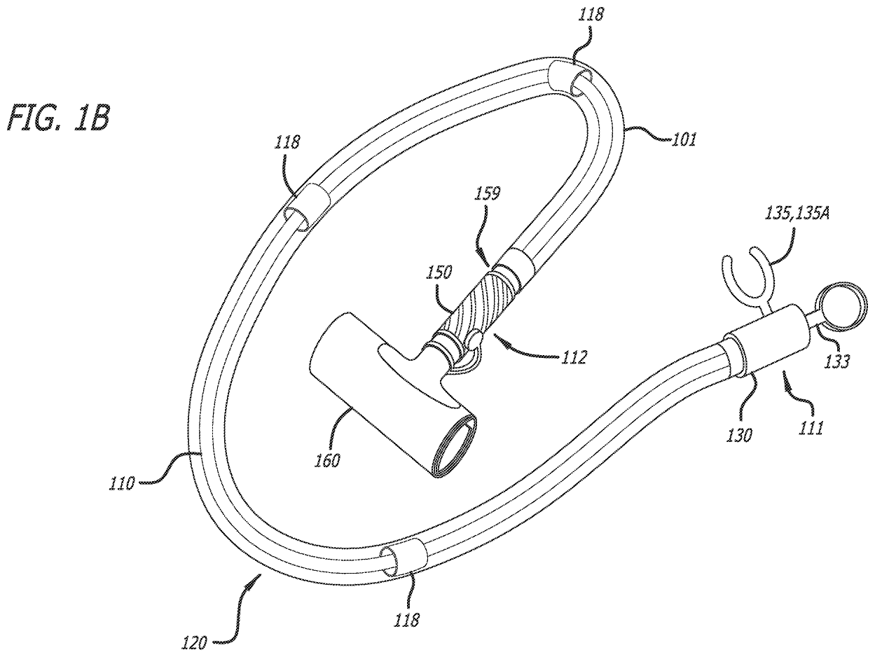

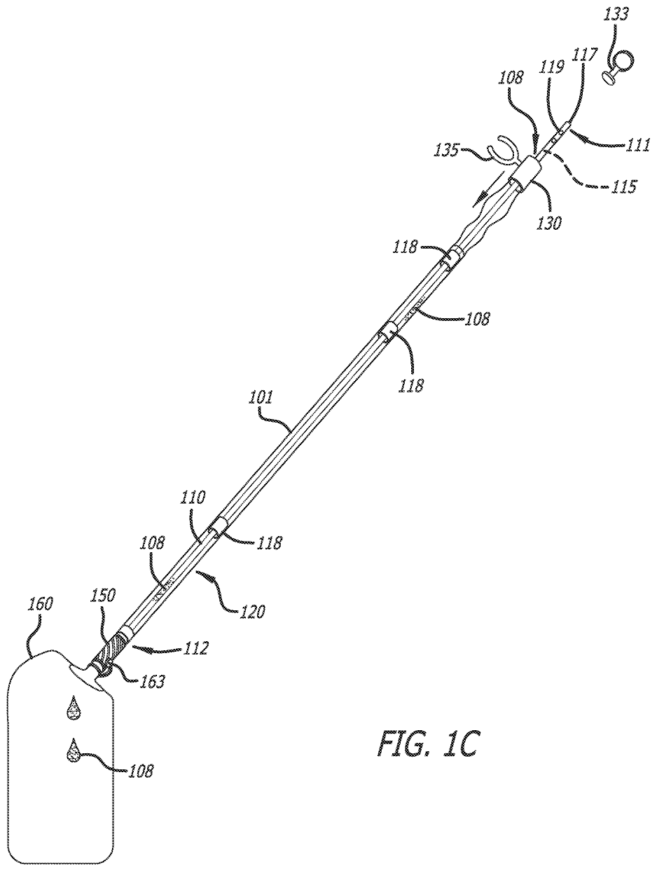

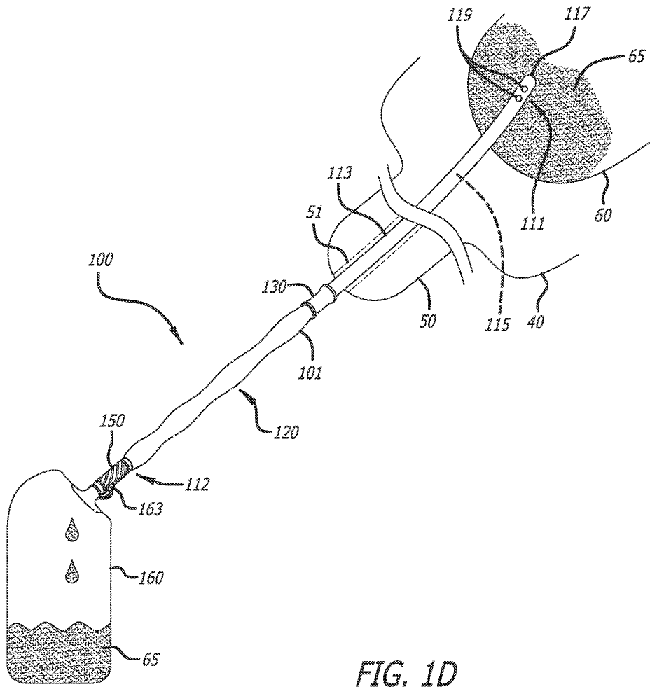

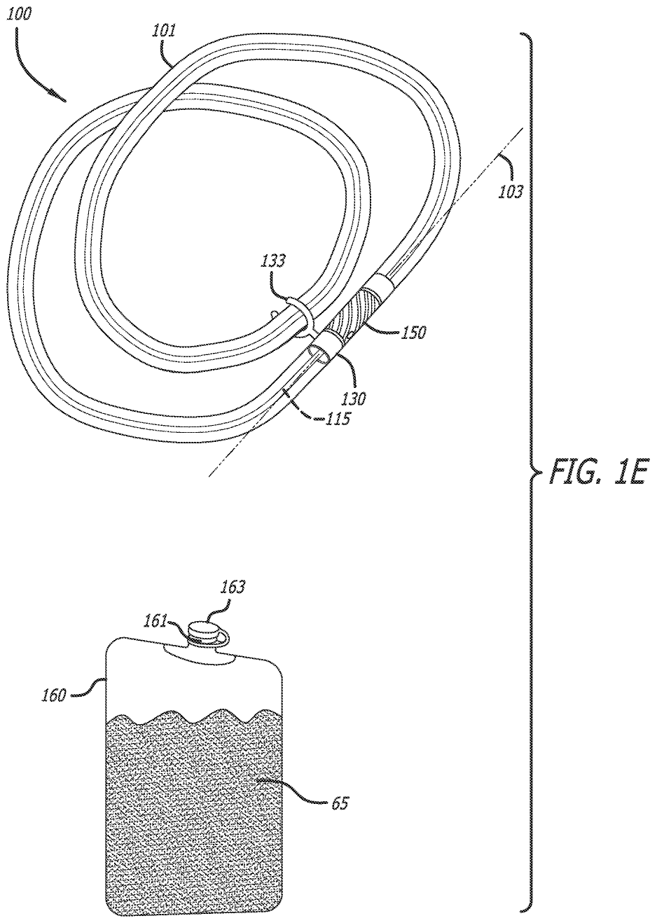

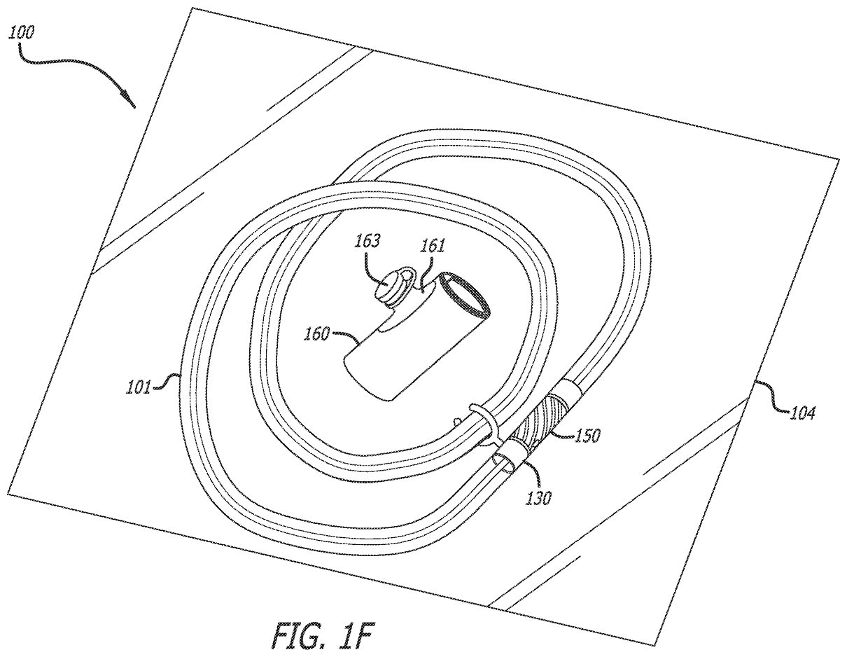

FIGS. 1A-1F illustrate a first embodiment of a urinary catheter system in various states of use. The urinary catheter system (system) 100 is generally configured for self-deployment by a user to drain the user's bladder of urine. The system 100 as illustrated is generally configured for use by a male user. However, the system 100 may be configured for use by a female user without departing from the features and functional aspects of the system 100 as shown and described below. The system 100 may be used by the user in various settings, such as at a medical facility, at a home, or at a public restroom, for example. In some instances, the system 100 may be used in the context of providing care to a patient. In such instances, the user, as referred to herein, may be a clinician providing the care as well as the patient receiving the care.

The system 100 is generally configured to be used by the user multiple times during a day. More specifically, the user may deploy multiple systems 100 (one at a time) during the day. As such, the user my keep and carry one or more systems 100 on his person, such as in a pocket of clothing or a purse, for example. As such, the system 100 may be disposed in pre-use packaging state having a compact shape consistent with placement of the system 100 within the pocket of clothing or purse. The system 100 is also configured to maintain a sterility of the system 100 in the pre-use packaging state.

In some instances, the user may be concerned about soiling clothing during the urine draining process, via the urine or a lubricant of the system, for example. Hence, the system 100 is configured for deployment while also inhibiting the spilling of a lubricant or urine of the system 100 on clothing or other items during use. Furthermore, the system 100 is generally configured to contain any residual urine within the catheter tubing or a collection bag after use.

As the system 100 is configured for self-deployment, the system 100 may be configured for ease in handling (i.e., gripping, orienting, guiding, or applying a force, etc.) by a user during deployment. As such, the system 100 may be configured for deployment by users having limited dexterity.

FIG. 1A illustrates a perspective front view of the system 100 in a pre-use packaging state. The system 100 may be disposed in the pre-use packaging state before use. The system 100 may be disposed within an outer package 104, such as a bag or pouch, for example. In some embodiments, the outer package 104 may be resealable. In other words, the outer package 104 may be configured to (i) sealably contain the system 100 before use and (ii) sealably contain the system 100 after use.

The system 100 generally includes a urinary catheter (catheter) 101 and a collection bag (bag) 160. During various stages of use, the bag 160 and the catheter 101 are coupled together. The catheter 101 generally includes a catheter tube 110 having a proximal end 112 and a distal end 111. The catheter tube 110 may be formed (e.g., extruded) of any suitable material, such as polyvinyl chloride (PVC), polyurethane, latex, or silicone, for example. A catheter lumen 115 extends along the catheter tube 110, where the catheter lumen 115 is sized to facilitate urine flow therethrough from the distal end 111 to the proximal end 112 during use. The catheter tube 110 includes an inlet section 113 extending proximally away from the distal end 111, and the inlet section 113 is configured for insertion into and advancement along a urethra of the user to the user's bladder. As such, the inlet section 113 defines a length sufficient to extend from the end of a penis to the bladder of the user. Similarly, the inlet section defines an outside diameter compatible with placement within the urethra. The catheter tube 110 also includes an outlet section 114 extending distally away from the proximal end 112. The outlet section 114 is configured for use outside of the body of the user.

The catheter 101 includes an inlet collar 130 coupled with the inlet section 113 to provide an insertion aid to the user. The inlet collar 130 is configured for gripping by the user during use. In some embodiments, the inlet collar 130 includes a cap (or plug) 133 that is configured to seal or close off the distal end 111 of the catheter 101 in the pre-use packaging state as further described below. In some embodiments, the cap 133 may include a pull ring 134 to facilitate removal of the cap 133 from the inlet collar 130 to break the seal. In some embodiments, the cap 133 may be configured for recoupling to the inlet collar 130 to reseal the distal end 111. The inlet collar 130 may also include an attachment member 135 as further described below. The inlet collar 130 and the cap 133 may be formed (e.g., molded) of any suitable material, such as polyvinyl chloride (PVC), or silicone, for example.

The catheter 101 also includes an outlet hub (hub) 150 defining a handle for the catheter 101 during use. The hub 150 is attached to the catheter tube 101 at the proximal end 112. The hub 150 includes gripping features 151 (e.g., protrusions, depressions, ribs, or toughs) disposed on an outside surface of the hub 150 to enable grasping of the hub 150 during use. The hub 150 defines a hub chamber 155. The hub chamber 155 is in fluid communication with the catheter lumen 115, i.e., the hub chamber 155 defines the catheter lumen 115 at the proximal end 112. The outlet hub 150 may be formed (e.g., molded) of any suitable material, such as polyvinyl chloride (PVC), or silicone, for example.

The system 100 further includes a sheath 120 extending along the catheter tube 110 from the inlet collar 130 to the hub 150. The sheath 120 defines a sheath lumen 125 and the catheter tube 110 is disposed within the sheath lumen 125. The sheath 120 (or more specifically a distal end of the sheath 120) is sealably attached to the inlet collar 130 and the sheath 120 is also sealably attached to the hub 150. A lubricant 108 is disposed within the sheath lumen 125, where the lubricant 108 is configured to enable sliding displacement of the inlet section 113 along the urethra during use. The sheath 120 may be formed of any plastic sheet material, such as high-density polyethylene (HDPE), low-density polyethylene (LDPE), or linear low-density polyethylene (LLDPE), for example.

The catheter 101 includes a number (e.g., 1, 2, 3, or more) of gripping members 118 disposed within the sheath lumen 125 to enable the user to manipulate the catheter tube 110 during use. The gripping members 118 may be dispersed along the catheter tube 110 and are slidably coupled with the catheter tube 110. In some embodiments, the gripping members 118 may be formed of a collar surrounding the catheter tube 110. The gripping members 118 are generally configured for selectively gripping the catheter tube 110 i.e., preventing slide-able displacement along the catheter tube 110. More specifically, the gripping members 118 may be crushable in response to a squeezing force applied by the user so that (i) the gripping members 118 may slidably displace along the catheter tube in the absence of the squeezing force, and (ii) the gripping members 118 may define co-movement of the catheter tube 110 with the gripping members 118 when the squeezing force is applied. As such, the gripping members 118 may be formed of an elastomeric material, such as silicone, for example, a foam material, such as rubber or polyurethane, for example, or any other suitable material. In some embodiments, one or more of the gripping members may be attached to the sheath 120.

The bag 160 is coupled with the catheter 101 in the pre-use packaging state as depicted in FIG. 1A. More specifically, the bag 160 includes an inlet hub 161 sealably coupled with the outlet hub 150 so that the catheter lumen 115 is in fluid communication with an interior of the bag 160. The bag 160 is folded or otherwise formed into a compact shape for placement within the outer package 104. The compact shape may also facilitate placement of the system 100 within the pocket or purse of the user. The bag 160 may be formed any suitable plastic sheet material, such as PVC, high-density polyethylene (HDPE), low-density polyethylene (LDPE), or linear low-density polyethylene (LLDPE), for example.

The system 100 includes a bag cap 163 configured for sealable placement onto the inlet hub 161 as further described below. The bag cap is attached to the bag 160 (e.g., the inlet hub 161) via a tether 162. The bag cap 163 is also selectively attachable to the outlet hub 150 to secure the outlet hub 150 to the inlet hub 161. As shown in FIG. 1A, the bag cap 163 is attached to the outlet hub 150 in the pre-use packaging state. The bag cap 163 may be formed (e.g., molded) of any suitable material, such as polyvinyl chloride (PVC), polyethylene, polypropylene, or silicone, for example.

FIG. 1B illustrates the system 100 in an unpackaged state, i.e., where the system 100 is removed from the outer package 104. As stated above, the inlet collar 130 includes the attachment member 135. The attachment member 135 is generally configured to maintain the catheter 101 in a compact shape, such as a coiled shape, for example. The attachment member 135 is configured for selective attachment to and detachment from the outlet hub 150. In some embodiments, attachment member 135 includes a clip 135A configured to engage a groove 159 of the outlet hub 150. In other embodiments, the attachment member 135 may be configured to attachingly engage with a portion of the catheter 101 other than the outlet hub 150, such as along the catheter tube 110, for example.

FIG. 1C illustrates the system 100 in a ready-to-use state. In the ready-to-use state, the catheter tube 110 is uncoiled, the bag 160 is unfolded, and the cap 133 is removed from the inlet collar 130. The inlet collar 130 is configured to slide along the catheter tube 110 away from the distal end 111. In the ready-to-use state, the inlet collar 130 is proximally displaced to expose a catheter tip 117. The catheter tip 117 includes a number (e.g., 1, 2, or more) of eyelets 119 of the catheter tube 110. The eyelets 119 define a distal opening of the catheter lumen 115 to allow urine to enter the catheter lumen 115 during use.

In some embodiments, a portion of the lubricant 108 may be disposed in the catheter lumen 115. In some instances, the user may orient the catheter 101 to allow the lubricant to drain from the catheter lumen 115 into the bag 160.

FIG. 1D illustrates the system 100 during use. The inlet collar 130 is proximally displaced to expose the inlet section 113 and the inlet section 113 is advanced through the urethra 53 so that the distal end 111 is disposed in the bladder 60 of the user 40. Urine 65 passes through the eyelets 119 into the catheter lumen 115. The urine 65 then flows from the catheter lumen 115 into the bag 160. The cap 163 is attached to the outlet hub 150 to maintain securement of the bag 160 to the catheter 101.

FIG. 1E illustrates the system 100 after use, i.e., after the urine 65 is drained from the user 40 and after the catheter 101 is removed from the user 40. The cap 133 is detached from the outlet hub 150. The bag cap 163 is sealably coupled with the inlet hub 161 to seal the urine 65 within the bag 160. In the illustrated embodiment, the outlet hub 150 and the inlet collar 130 are configured to sealably coupled together. As such, FIG. 1E depicts the inlet collar 130 coupled with the outlet hub 150 to seal any residual urine 65 within the catheter lumen 115. The coupling of the inlet collar 130 to the outlet hub 150 along a common longitudinal axis 103 may facilitate a compact shape (e.g., a coiled shape) of the catheter 101 for redisposition of the catheter 101 within the outer package 104 (see FIG. 1A). The attachment member 133 may be optionally configured for attachment to the catheter tube 110 to further define the compact shape for redisposition of the catheter 101 within the outer package 104.

In use, the user may drain the urine 65 from the bag 160 by first separating the bag cap 163 from the inlet hub 161. The user may then recouple the bag cap 163 to the inlet hub 161 to seal any residual urine 65 within the bag 160. The user may then fold or otherwise form the bag 160 into a compact shape for redisposition of the bag 160 within the outer package 104.

FIG. 1F illustrates the system 100 in a disposal state. The catheter 101 is disposed in the compact shape with the inlet collar 130 coupled with the outlet hub 150. The bag 160 is formed into the compact shape with the bag cap 163 coupled with the inlet hub 161. The bag 160 and the catheter 101 are redisposed in the outer package 104.

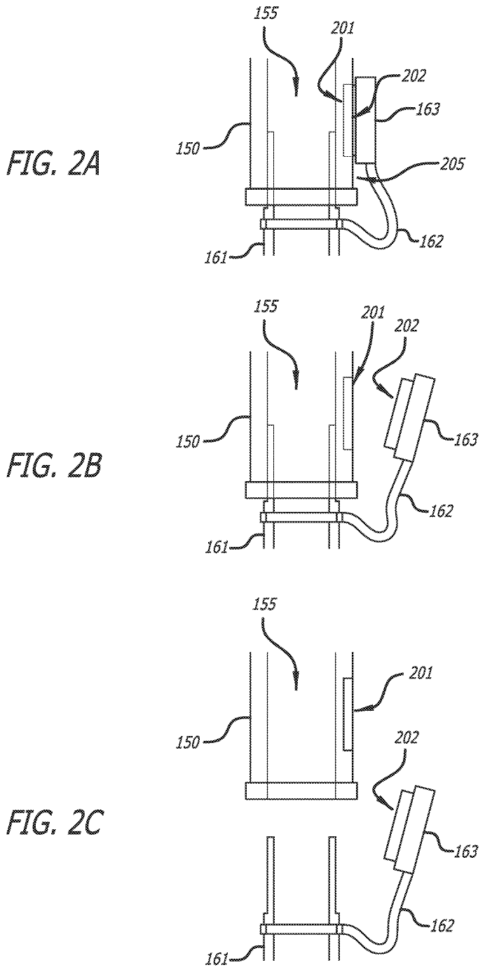

FIGS. 2A-2C illustrates detailed views of the inlet hub 161 and the outlet hub 150 in various states of coupling. FIG. 2A illustrates the inlet hub 161 and the outlet hub 150 in the coupled state, such as the sates illustrated in FIGS. 1A-1D. In the illustrated embodiment, the inlet hub 161 is disposed within the chamber 155 of the outlet hub 150. In other embodiments, inlet hub 161 may be coupled with the outlet hub 150 via other suitable arrangements of the inlet hub 161 and the outlet hub 150, such as a portion of the outlet hub 150 disposed within the inlet hub 161, for example. The inlet hub 161 may form an interference fit with the outlet hub 150 to define the seal between the inlet hub 161 and the outlet hub 150.

As depicted in FIGS. 1A-1D, the bag cap 163 may be attached to the outlet hub 150 along a side wall 205 of the outlet hub 150. In the illustrated embodiment, the outlet hub 150 includes a receiving depression 201 along the side wall 205, and the bag cap 163 includes a corresponding protrusion 202. In other embodiments, the outlet hub 150 may include the protrusion 202 and the bag cap 163 may include the depression 201. The receiving depression 201 and the corresponding protrusion 202 are configured to define an interference fit, such as a snap fit, for example. In more general terms, the receiving depression 201 and the corresponding protrusion 202 are configured to prevent inadvertent separation of the bag cap 163 from the outlet hub 150.

As also depicted in FIGS. 1A-1D, the bag cap 163 is attached to the inlet hub 161 via the tether 162. The tether 162 may be sized (i.e., may define a length) so that inlet hub 161 sealably engaged with the outlet hub 150 when the bag cap 163 is attached to the outlet hub 150.

FIG. 2B illustrates the inlet hub 161 and the outlet hub 150 in a partially decoupled state. The bag cap 163 is detached from the outlet hub 150 while the inlet hub 161 remains sealably coupled with the outlet hub 150. As shown, the protrusion 202 is removed from the depression 201.

FIG. 2C illustrates the inlet hub 161 and the outlet hub 150 in a fully decoupled state. The bag cap 163 is detached from the outlet hub 150 and the inlet hub 161 is separated from the outlet hub 150. As shown, the tether 162 is sufficiently short to preclude detachment of the inlet hub 161 from the outlet hub 150 when the protrusion 202 is disposed within the depression 201.

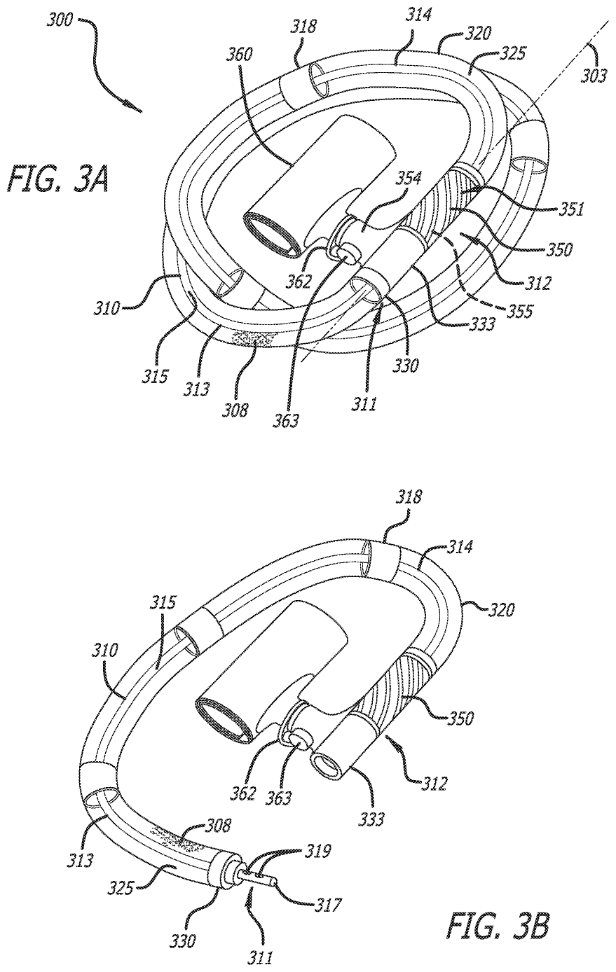

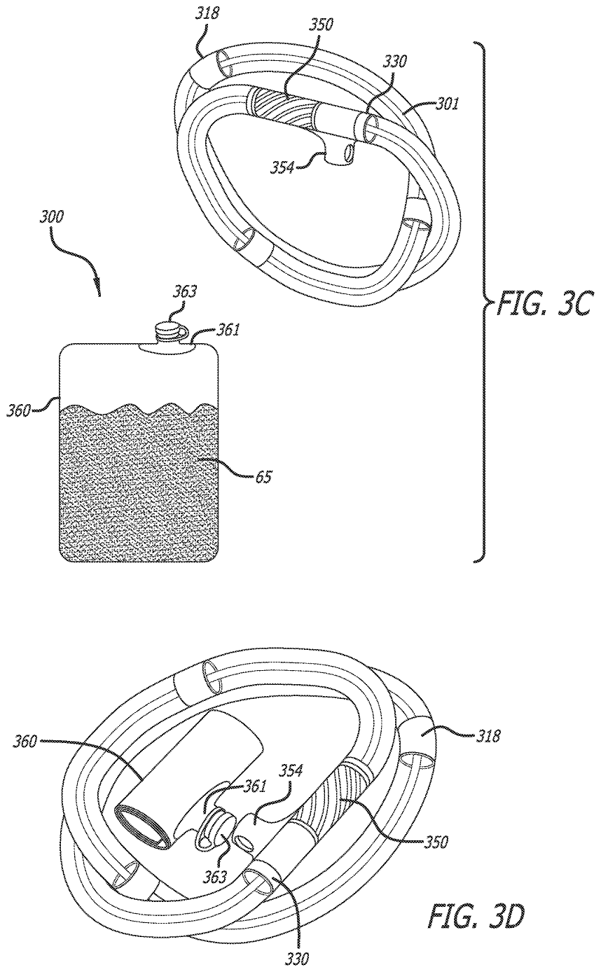

FIGS. 3A-3D illustrate another embodiment of a urinary catheter system 300 that can, in certain respects, resemble components of the urinary catheter system 100 described in connection with FIGS. 1A-2C. It will be appreciated that all the illustrated embodiments may have analogous features. Accordingly, like features are designated with like reference numerals, with a leading digit of “3.” For instance, the catheter is designated as “101” in FIGS. 1A-2C, and an analogous catheter is designated as “301” in FIGS. 3A-3D. Relevant disclosure set forth above regarding similarly identified features thus may not be repeated hereafter. Moreover, specific features of the urinary catheter system 100 and related components shown in FIGS. 1A-2C may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the urinary catheter system of FIGS. 3A-3D. Any suitable combination of the features, and variations of the same, described with respect to the urinary catheter system 100 and components illustrated in FIGS. 1A-2C can be employed with the urinary catheter system and components of FIGS. 3A-3D, and vice versa.

FIG. 3A illustrates a perspective front view of the system 300 in a pre-use packaging state. The system 300 may be disposed in the pre-use packaging state before use. The system 300 may be disposed within an outer package (not shown but similar to the outer package 104 of FIG. 1A).

The system 300 generally includes a urinary catheter (catheter) 301 and a collection bag (bag) 360. During various stages of use, the bag 360 and the catheter 301 are coupled together. The catheter 301 generally includes a catheter tube 310 having a proximal end 312 and a distal end 311. The catheter tube 310 may be formed of any suitable thermoplastic material via an extrusion process. A catheter lumen 315 extends along the catheter tube 310, where the catheter lumen 315 is sized to facilitate urine flow therethrough from the distal end 311 to the proximal end 312 during use. The catheter tube 310 includes an inlet section 313 extending proximally away from the distal end 311, and the inlet section 313 is configured for insertion into and advancement along a urethra of the user to the bladder. As such, the inlet section 313 defines a length sufficient to extend from the end of a penis to the bladder of the user. Similarly, the inlet section defines an outside diameter compatible with placement within the urethra. The catheter tube 310 also includes an outlet section 314 extending distally away from the proximal end 312. The outlet section 314 is configured for use outside of the body of the user.

The catheter 301 includes an inlet collar 330 coupled with the inlet section 313 to also provide an insertion aid to the user. The inlet collar 330 is configured for gripping by the user during use. The catheter 301 includes a cap 333 that is it is integrated into an outlet hub 350. The cap 333 is configured to couple with the inlet hub 330 to seal or close off the distal end 311 of the catheter 301 in the pre-use packaging state as further described below. In some embodiments, the cap 333 and the inlet hub 330 define a common longitudinal axis 303 when coupled together. During use, the inlet collar 330 is separated from cap 333 to break the seal. In some embodiments, the cap 333 may be configured for recoupling to the inlet collar 330 to reseal the distal end 311.

The catheter 301 also includes the outlet hub (hub) 350 defining a handle for the catheter 301 during use. The hub 350 is attached to the catheter tube 301 at the proximal end 312. The hub 350 includes gripping features 351 (e.g., protrusions, depressions, ribs, or toughs) disposed on an outside surface of the hub 350 to enable grasping of the hub 350 during use. The 350 defines a hub chamber 355. The hub chamber 355 is in fluid communication with the catheter lumen 315, i.e., the hub chamber 355 defines the catheter lumen 315 at the proximal end 312.

The system 300 further includes a sheath 320 extending along the catheter tube 310 from the inlet collar 330 to the hub 350. The sheath 320 defines a sheath lumen 325 and the catheter tube 310 is disposed within the sheath lumen 325. The sheath 320 (or more specifically a distal end of the sheath 320) is sealably attached to the inlet collar 330, and the sheath 320 is also sealably attached to the hub 350. A lubricant 308 is disposed within the sheath lumen 325, where the lubricant 308 is configured to enable sliding displacement of the inlet section 313 along the urethra during use.

The catheter 301 includes a number (e.g., 1, 2, 3, or more) of gripping members 318 disposed within the sheath lumen 325 to enable the user to manipulate the catheter tube 310 during use. The gripping members 318 may be dispersed along the catheter tube 310 and are slidably coupled with the catheter tube 310. In some embodiments, the gripping members 318 may be formed of a collar surrounding the catheter tube 310. The gripping members 318 are generally configured for selectively gripping the catheter tube 310 i.e., preventing slide-able displacement along the catheter tube 310. More specifically, the gripping members 318 may be crushable in response to a squeezing force applied by the user so that (i) the gripping members 318 may slidably displace along the catheter tube in the absence of the squeezing force, and (ii) the gripping members 318 may define co-movement of the catheter tube 310 with the gripping members 318 when the squeezing force is applied. In some embodiments, one or more of the gripping members may be attached to the sheath 320.

The bag 360 is coupled with the catheter 301 in the pre-use packaging state as depicted in FIG. 3A. More specifically, the bag 360 includes an inlet hub 361 sealably coupled with a side port 354 of the outlet hub 350 so that the catheter lumen 315 is in fluid communication with an interior of the bag 360. The bag 360 is folded or otherwise formed into the compact shape for placement within the outer package. The compact shape may also facilitate placement of the system 300 within the pocket or purse of the user.

The system includes a bag cap 363 configured for sealable placement onto the inlet hub 361 as further described below. The bag cap 363 is attached to the bag 360 (e.g., the inlet hub 361) via a tether 362. The bag cap 363 is also selectively attachable to the side port 354 to secure the outlet hub 350 to the inlet hub 361. As shown in FIG. 3A, the bag cap 363 is attached to the side port 354 in the pre-use packaging state.

FIG. 3B illustrates the system 300 in a ready-to-use state. In the ready-to-use state, the catheter tube 310 is uncoiled, and inlet collar 330 is decoupled from the cap 333. The bag is ready to be unfolded for use. The inlet collar 330 is configured to slide along the catheter tube 310 away from the distal end 311. In the ready-to-use state, the inlet collar 330 is proximally displaced to expose a catheter tip 317. The catheter tip 317 includes a number (e.g., 1, 2, or more) of eyelets 319 of the catheter tube 310. The eyelets 319 define a distal opening of the catheter lumen 315 to allow urine to enter the catheter lumen 315 during use.

FIG. 3C illustrates the system 300 after use, i.e., after the urine 65 is drained from the user 40 (see FIG. 1D) and after the catheter 301 is removed from the user 40. The bag cap 363 is detached from the side port 354. The bag cap 363 is sealably coupled with the inlet hub 361 to seal the urine 65 within the bag 360. The inlet collar 330 is coupled with the outlet hub 350 via the integrated cap 333 to seal any residual urine 65 within the catheter lumen 315. The coupling of the inlet collar 330 to the outlet hub 350 may redefine the compact shape (e.g., a coiled shape) of the catheter 301 for redisposition of the catheter 301 within the outer package.

In use, the user may drain the urine 65 from the bag 360 by first separating the bag cap 363 from the inlet hub 361. The user may then recouple the bag cap 363 to the inlet hub 361 to seal any residual urine 65 within the bag 360. The user may then fold or otherwise from the bag 360 into the compact shape for redisposition of the bag 360 within the outer package.

FIG. 3D illustrates the system 300 in a disposal state. The catheter 301 is disposed in the compact shape with the inlet collar 130 coupled with the outlet hub 150 via the integrated cap 333. The bag 360 is formed into the compact shape with the bag cap 363 coupled with the inlet hub 361.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urinary catheter system, comprising:
a catheter comprising:
a catheter tube defining a catheter lumen extending between a distal end and a proximal end of the catheter, the catheter tube configured for insertion into a bladder of a user;
an outlet hub coupled with the catheter tube at the proximal end, the outlet hub defining a handle configured for manipulation of the catheter during use; and
a collection bag comprising:
an inlet hub defining an opening of the collection bag; and
an engagement member tethered to the inlet hub, the engagement member configured to selectively couple with the inlet hub to sealably close off the opening, thereby sealing the collection bag, and decouple from the inlet hub to provide for access to contents of the collection bag, wherein:
the inlet hub is coupled with the outlet hub at a proximal opening of the outlet hub to define a fluid flow path between the catheter lumen and an interior of the collection bag, and
the engagement member is further configured to selectively attach to the outlet hub to prevent separation of the inlet hub from the outlet hub, and detach from the outlet hub to allow separation of the collection bag from the catheter.

2. The system of claim 1, wherein the engagement member is configured to attach to the outlet hub along a side wall of the outlet hub.

3. The system of claim 1, wherein:
the engagement member includes a protrusion, and
the outlet hub includes a corresponding depression configured to receive the protrusion to define the attachment of the engagement member to the outlet hub.

4. The system of claim 1, wherein:
the catheter further comprises an inlet collar disposed at the distal end of the catheter, and
the inlet collar is attached to the outlet hub to define a pre-use packaging state of the catheter.

5. The system of claim 4, wherein the inlet collar includes a clip configured to attach the inlet collar to the outlet hub in the pre-use packaging state.

6. The system of claim 4, wherein the inlet collar is configured to couple with the outlet hub at the proximal opening to define a post-use packaging state of the catheter.

7. The system of claim 4, wherein:
the catheter further comprises a sheath extending between the outlet hub and the inlet collar, the sheath defining a sheath lumen; and
the catheter tube is disposed within the sheath lumen.

8. The system of claim 7, wherein the catheter further comprises a number of gripping members disposed within the sheath lumen, each gripping member configured to:
grip the catheter tube in response to a squeezing force applied to the gripping member by a user, and
slide along the catheter tube in the absence of the gripping force.

9. The system of claim 1, wherein the catheter further includes a cap coupled with the inlet collar, the cap configured to close off a distal end of the inlet collar in the pre-use packaging state.

10. A urinary catheter system, comprising:
a catheter comprising:
a catheter tube defining a catheter lumen extending between a distal end and a proximal end of the catheter, the catheter tube configured for insertion into a bladder of a user;
an outlet hub coupled with the catheter tube at the proximal end, the outlet hub defining a handle configured for manipulation of the catheter during use; and
an inlet collar disposed at the distal end; and
a collection bag comprising an inlet hub defining an opening of the collection bag, wherein:
the inlet hub is coupled with the outlet hub at a side port of the outlet hub to define a fluid flow path between the catheter lumen and an interior of the collection bag, and
the inlet collar is coupled with the outlet hub to define a pre-use packaging state.

11. The system of claim 10, wherein the inlet collar and the outlet hub define a common longitudinal axis in the pre-use packaging state.

12. The system of claim 10, wherein the catheter further comprises an engagement member tethered to the inlet hub, the engagement member configured to selectively:
couple with the inlet hub to sealably close off the opening, thereby sealing the collection bag; and
attach to the outlet hub to prevent separation of the inlet hub from the outlet hub.

13. The system of claim 10, wherein:
the catheter further comprises a sheath extending between the outlet hub and the inlet collar, the sheath defining a sheath lumen; and
the catheter tube is disposed within the sheath lumen.

14. The system of claim 13, wherein the catheter further comprises a number of gripping members disposed within the sheath lumen, each gripping member configured to:
grip the catheter tube in response to a squeezing force applied to the gripping member by a user, and
slide along the catheter tube in the absence of the gripping force.

15. A method for draining urine from a bladder of a user, comprising:
providing a catheter system, comprising:
a catheter coupled with a collection bag, the catheter including a catheter tube defining a catheter lumen, wherein:
the catheter includes an outlet hub at a proximal end of the catheter and the collection bag includes an inlet hub, the outlet hub coupled with the inlet hub such that the catheter lumen is in fluid communication with an interior of the collection bag;
the catheter is attached to the collection bag via a tether; and
the outlet hub is attached to an inlet collar of the catheter, the inlet collar disposed at distal end of the catheter;
detaching the outlet hub from the inlet collar;
advancing the catheter tube along a urethra to the bladder of the user to access urine within the bladder;
draining the urine from the bladder, the urine flowing along the lumen of the catheter into the interior of the collection bag;
detaching the tether from the catheter;
separating the inlet hub from the outlet hub; and
draining the urine from the interior of the collection bag.

16. The method of claim 15, wherein:
the tether includes a bag cap attached thereto,
the catheter attached to the bag via the tether includes the bag cap attached to the outlet hub, and
detaching the tether from the catheter includes detaching the bag cap from the outlet hub.

17. The method of claim 15, further comprising attaching the bag cap to the inlet hub to seal the urine within the collection bag.

18. The method of claim 15, wherein advancing the catheter tube includes proximally displacing the inlet collar along the catheter tube.

19. The method of claim 15, further comprising:
distally displacing the inlet collar along the catheter tube after draining the urine, and
attaching the inlet collar to the outlet hub to define a disposal state of the catheter.

20. The method of claim 15, wherein the catheter further includes:
a sheath extending along the catheter tube, the catheter tube disposed within a lumen of the sheath; and
a number of gripping members disposed within the lumen of the sheath, each gripping member configured to:
grip the catheter tube in response to a squeezing force applied to the gripping member by a user, and
slide along the catheter tube in the absence of the gripping force,
wherein the method further comprises advancing the catheter tube includes applying a squeezing force to at last one gripping member.
